(19) United States
(12) Patent Application Publication
(10) Pub. No.: US 2018/0177910 A1
(43) Pub. Date: Jun. 28, 2018

(54) WOUND DRESSING COMPRISING COPPER

(71) Applicant: Medicore Global LLC, Simpsonville, SC (US)

(72) Inventor: Richard Charles Zatcoff, Greer, SC (US)

(21) Appl. No.: 15/736,430

(22) PCT Filed: Jun. 23, 2016
(86) PCT No.: PCT/US2016/038884

§ 371 Date: Dec. 14, 2017

Related U.S. Application Data
(60) Provisional application No. 62/184,463, filed on Jun. 25, 2015.

(51) Int. Cl. A61L 15/44; A61K 33/34; A61L 15/58

(52) U.S. Cl. CPC A61L 15/44; A61L 2300/404; A61L 15/58; A61K 33/34

(57) ABSTRACT

A therapeutic wound dressing which includes a textile material and copper, wherein the copper is present in an effective amount to stimulate wound healing and to restrict growth of microorganisms. At least some of the copper is proximate exterior surfaces of at least some of the textile fibers. The wound dressing is configured such that the dressing may be held against a wound in human or animal tissue, thereby exposing the wound to copper.

### CROSS-REFERENCE TO RELATED APPLICATIONS

This application claims priority to U.S. Provisional Patent Application No. 62/184,463, filed Jun. 25, 2015.

### FIELD OF THE INVENTION

The present invention relates to a wound dressing comprising a textile and copper. The copper is present in an effective amount to promote wound healing and retard microbial growth. The invention further relates to the use of dressing for treatment of wounds.

### BACKGROUND OF THE INVENTION

Some wound dressings have included silver for antimicrobial purposes. However, there can be complications associated with the use of wound dressings that include silver. There is a desire for wound dressings that provide a new balance of properties.

### SUMMARY OF THE INVENTION

The invention relates to wound dressings and, more particularly, to wound dressings that include copper. In one aspect of the invention, the wound dressing comprises a textile material and copper, wherein the copper is present in an effective amount for both stimulating wound healing and restricting growth of microorganisms.

In another aspect of the invention, the wound dressing is comprised of a substrate and a textile material mounted to the substrate, the textile material comprising fibers and copper, wherein at least some of the copper is proximate exterior surfaces of at least some of the fibers, and wherein the copper is present in an effective amount to stimulate wound healing and to restrict growth of microorganisms.

In another aspect of the invention, the textile material is a nonwoven textile fabric. In another aspect, the textile material is a pad that is thicker than the substrate. In some aspects, the nonwoven textile fabric is a needled nonwoven textile fabric. In some aspects, the substrate is an apertured substrate. In some aspects, the substrate is an apertured nonwoven textile fabric. In a further aspect, the substrate is laminated to the textile material.

In a further aspect, the substrate is a backing wherein at least one marginal portion of the backing extends outwardly beyond a periphery of the textile material, the wound dressing further comprises adhesive material on the at least one marginal portion of the backing, and the adhesive material is configured for at least partially mounting the wound dressing to animal tissue. In some aspects, the at least one marginal portion of the backing extends at least partially around the textile material and the adhesive material extends at least partially around the textile material.

In some aspects, the wound dressing comprises a nonwoven textile fabric, the nonwoven textile fabric further comprising fibers and copper, wherein at least some of the copper is proximate exterior surfaces of at least some of the fibers, and the copper is present in an effective amount to stimulate wound healing and to restrict growth of microorganisms. In some aspects, the nonwoven textile fabric is a needled nonwoven textile fabric. In other aspects, the wound dressing further comprises a substrate mounted to a nonwoven textile fabric. In some aspects, the nonwoven textile fabric is thicker than the substrate. In some aspects, the substrate is apertured. In some aspects, the substrate is an apertured nonwoven textile fabric.

In some aspects, the wound dressing comprises a nonwoven textile fabric, the nonwoven textile fabric further comprising fibers and copper, wherein at least some of the copper is proximate exterior surfaces of at least some of the fibers, and the copper is present in an effective amount to stimulate wound healing and to restrict growth of microorganisms, and further comprising a substrate mounted to the nonwoven textile fabric, wherein the substrate is a backing. In some aspects, at least one marginal portion of the backing extends outwardly beyond a periphery of the nonwoven textile fabric, the wound dressing further comprises adhesive material on the at least one marginal portion of the backing, and the adhesive material is configured for at least partially mounting the wound dressing to animal tissue.

In another aspect, the at least one marginal portion of the backing extends at least partially around the nonwoven textile fabric and the adhesive material extends at least partially around the nonwoven textile fabric. In some aspects, the substrate is laminated to the nonwoven textile fabric. In some aspects, the nonwoven textile fabric may be laminated to an apertured backing.

In an additional aspect, a method for treating a wound is provided, comprising applying a wound dressing according to the present invention to the wound. In some aspects, the wound dressing may be engaged against a wound such that the copper is available to both stimulate healing of the wound and restrict growth of microorganisms in the wound.

### BRIEF DESCRIPTION OF THE DRAWINGS

Aspects of the invention will be further described by reference to the accompanying drawings, which are schematic and not necessarily drawn to scale. The drawings are for example only, and should not be construed as limiting the invention.

FIG. 1 is a top plan view of a wound dressing, in accordance with a first aspect of the invention.

FIG. 2 is a cross-sectional view taken along line 2-2 of FIG. 1, wherein thicknesses are exaggerated in order to clarify the view.

FIG. 3 is a greatly enlarged, isolated view of a pair of entangled fibers of a nonwoven textile pad of the wound dressing of FIG. 1.

FIG. 4 is a further enlarged, cross-sectional view taken along line 4-4 of FIG. 3.

FIG. 5 is an enlarged, isolated plan view of a portion of a backing material of the wound dressing of FIG. 1.

FIG. 6 is a cross-sectional view of the wound dressing of FIG. 1 in contact with injured animal tissue, in accordance with an example of a method of use.

FIG. 7 is a bottom plan view of a wound dressing, in accordance with a second aspect of the invention.

FIG. 8 is a cross-sectional view taken along line 8-8 of FIG. 7, wherein thicknesses are exaggerated in order to clarify the view.

### DETAILED DESCRIPTION OF THE INVENTION

Exemplary embodiments are described below and illustrated in the accompanying drawings, in which like numerals refer to like parts throughout the several views. The embodiments described provide examples and should not be interpreted as limiting the scope of the invention. Other embodiments, and modifications and improvements of the described embodiments, will occur to those skilled in the art and all such other embodiments, modifications and improvements are within the scope of the present invention. For example, features illustrated or described as part of one embodiment can be used in the context of another embodiment to yield a further embodiment, and these further embodiments are within the scope of the present invention.

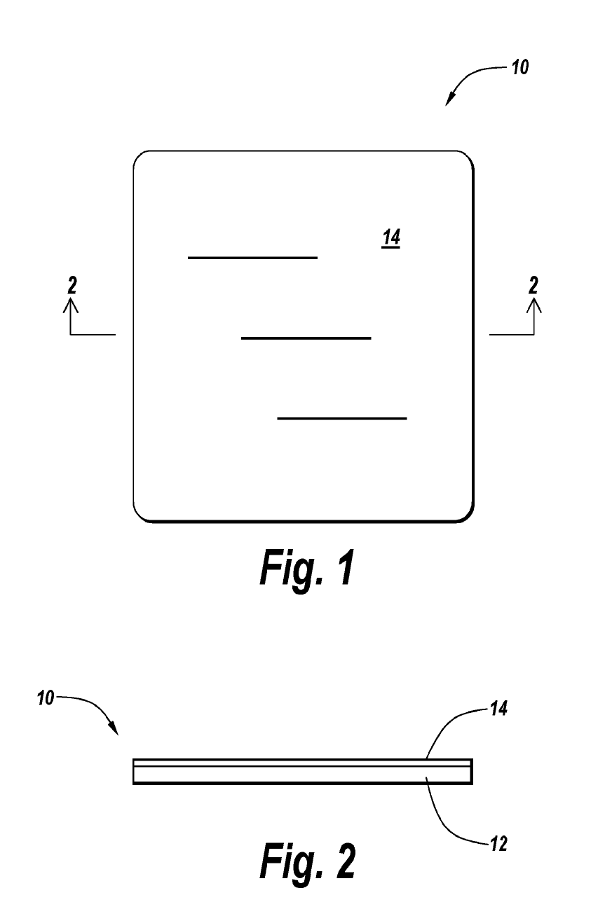

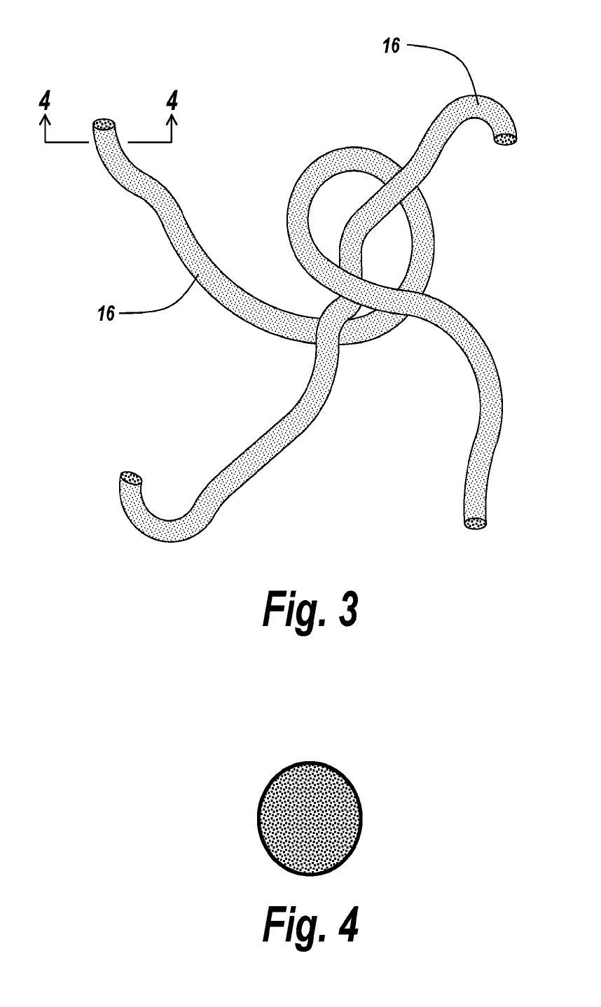

Referring now in greater detail to the drawings, FIGS. 1 and 2 illustrate a wound dressing 10 of a first embodiment of this disclosure. The wound dressing 10 may comprise, consist essentially of, or consist of nonwoven fibrous textile material that is in the form of a pad 12 and is fixedly mounted to a substrate or backing 14. The pad 12 may be absorbent. FIG. 3 is an enlarged, isolated view of a representative pair of entangled polymeric staple fibers 16 of the pad 12. The pad 12 may be a needled nonwoven textile fabric that may comprise, consist essentially of, or consist of numerous entangled polymeric staple fibers 16 that include and/or have associated therewith copper. In one implementation of the first embodiment, the copper is present in an effective amount sufficient to stimulate wound healing. In another implementation of the first embodiment, the copper is present in an effective amount sufficient to at least restrict growth of (e.g., kill) microorganisms. In other implementations of the first embodiment, the copper is present in an effective amount sufficient to both stimulate wound healing and at least restrict growth of microorganisms.

In some implementations, at least particles of the copper may be present proximate the exterior surfaces of at least some of the fibers 16 of the pad 12. In other implementations, the copper particles may be incorporated into, and present at the exterior surfaces of, at least some of the fibers 16 of the pad 12. The copper particles are schematically represented by stippling (i.e., dots and/or flicks) in FIGS. 3 and 4.

The fibers 16 of the pad 12 may have been cut from filaments manufactured by extruding a melted polymeric material. The fine copper particles may be, or may be part of, a melt additive that is mixed into and extruded with the base polymer material of the filaments. As a result, the copper particles may be distributed substantially throughout the polymer matrices of the fibers 16 of the pad 12, including copper particles being exposed at the exterior surfaces of the fibers of the pad. Accordingly, the fibers 16 of the pad 12 may be referred to as matrix fibers, or they may be more generally referred to as composite fibers 16. As another example, the composite fibers 16 of the pad 12 may have been cut from filaments manufactured by extruding a melted polymeric material, wherein the copper or copper particles may be chemically and/or mechanically added to the filaments and/or composite fibers after the extrusion, and thereafter the color of the filaments and/or composite fibers may be modified using suitable colorant(s). The base polymer material of the matrix or composite fibers 16 may be polyester, nylon or other suitable materials.

In the composite fibers 16 (and, thus, the pad 12) the copper (e.g., copper particles) may be present in a range of from about 0.1% to about 0.4% by weight of the composite fibers, any subranges therebetween, or in an amount of about 0.2% by weight of the composite fibers; or in a range of from about 30 parts per million (“ppm”) to about 120 ppm of the composite fibers, or any subranges therebetween.

In one implementation, the copper (e.g., copper particles) may be present in the composite fibers 16 (and, thus, the pad 12) in an amount of at least 0.01% by weight of the composite fibers, illustratively, about 0.01% to about 4% by weight of the composite fibers, about 0.05% to about 1% by weight of the composite fibers, and about 0.1% to about 0.4% by weight of the composite fibers.

In some implementations, the copper (e.g., copper particles) may be present in the composite fibers 16 (and, thus, the pad 12) in an amount of at least 0.1%, or at least about 0.1%, by weight of the composite fibers.

In some implementations, the copper (e.g., copper particles) may be present in the composite fibers 16 (and, thus, the pad 12) in an amount of at least 3 ppm of the composite fibers, illustratively, about 3 ppm to about 1000 ppm of the composite fibers, about 10 ppm to about 500 ppm of the composite fibers, and about 30 ppm to about 120 ppm of the composite fibers.

In other implementations, the copper (e.g., copper particles) may be present in the composite fibers 16 (and, thus, the pad 12) in an amount of at least 30 ppm, or at least about 30 ppm, of the composite fibers.

Alternatively, in addition to including the composite fibers 16, the pad 12 may further include any other suitable fibers, such as, but not limited to, cotton fibers. Suitable fibers 16 for the pad 12 (e.g., composite fibers and/or other suitable fibers) include, for example, copper impregnated polyester or polyamide)(Cu+STAT®).

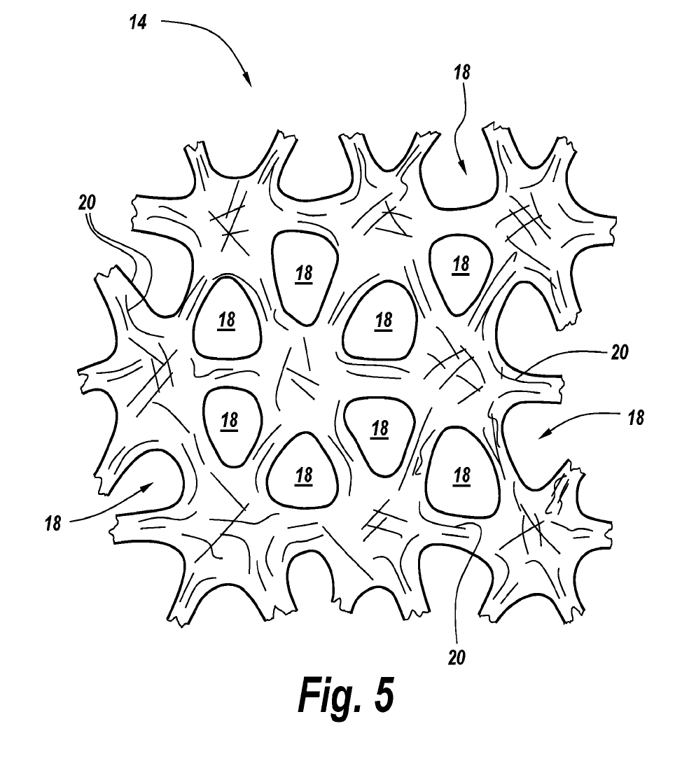

The backing 14 may be a suitable substrate of the type that is typically included in conventional wound dressings. More specifically, FIG. 5 is an enlarged, very schematic view of a portion of the backing 14 of the first embodiment of the wound dressing 10. As shown in FIG. 5, the backing 14 is in the form of an apertured substrate. More specifically, the backing 14 may be an apertured nonwoven textile fabric that is thinner than, and has less loft than, the pad 12. In the first embodiment, the holes 18 (e.g., apertures) extend completely through the backing 14 and are cooperatively defined by fibers 20 of the backing. Only a few of the fibers 20 of the backing 14 are schematically shown in FIG. 5.

The fibers 20 of the backing 14 may comprise, consist essentially of, or consist of the same material that is used as the base polymer material of the composite fibers 16, so that the pad 12 and backing can be directly laminated to one another by applying pressure and heat. For example, the pad 12 and the backing 14 may be fixedly connected to one another by way of direct connections between the pad and the backing. These direct connections may comprise, consist essentially of, or consist of portions of respective composite fibers 16 being thermally fused to portions of respective fibers 20 of the backing 14, such as by way of a thermal lamination process. Alternatively, if the pad 12 and backing 14 comprise materials that may not be readily directly thermally bonded to one another, the wound dressing 10 may further include adhesive material, a tie layer and/or other suitable materials positioned between the pad and backing, such as for fixedly mounting the pad to the backing. Other additional materials may also be included in the wound dressing 10 for other reasons.

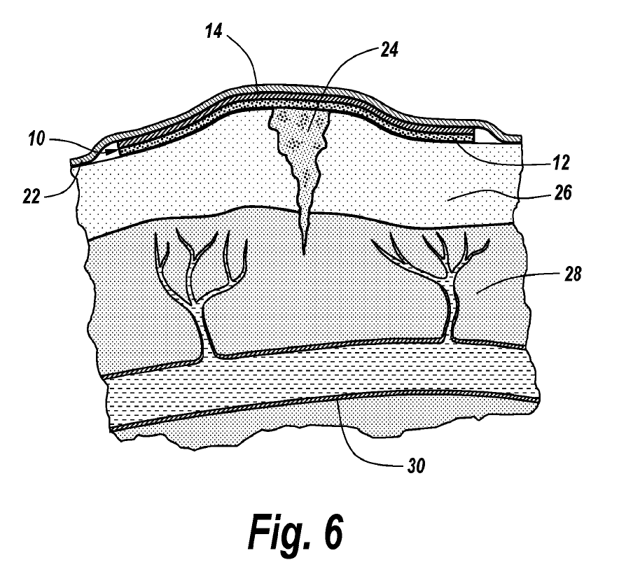

FIG. 6 illustrates the wound dressing 10 being held against a wound 24 (e.g., cut or burn) in animal tissue (i.e., human tissue or veterinary tissue), such as with one or more pieces of conventional adhesive tape 22 and/or other suitable devices, in accordance with an example of a method of use. As an example, the wound 24 is shown extending through the epidermis 26 and into the dermis 28, and in the vicinity of blood vessels extending outwardly from an artery 30. In the example shown in FIG. 6, the lower surface of the pad 12 is engaged against (e.g., in opposing face-to-face contact with) the wound 24. Therefore, at least some of the copper or copper particles positioned at or proximate the exterior of the composite fibers 16 are exposed to the wound 24 and associated fluids. As a result, at least some of the copper or copper particles of the pad 12 function both as an antimicrobial agent and a wound-healing agent for the wound. A variety of different types and configurations of wounds 24 are within the scope of this invention.

Generally regarding the antimicrobial aspects of the copper or copper particles of the wound dressing 10, copper can be bactericidal, virucidal, fungicidal, molluscicidal and/or acaricidal. Copper can be nonselective in the organisms that it is able to kill, including both gram-negative and gram-positive bacteria. It is believed that the effectiveness of copper against bacteria may be related to its multiple mechanisms. For example, copper can not only disrupt the bacterial cell membrane, but can also then attack the internal operating mechanisms of the bacteria itself, interfering with cell integrity and/or metabolism, leading to DNA degradation and/or cell death.

Regarding the wound-healing aspects of the copper or copper particles of the wound dressing 10, copper is typically present as either copper (Cu)⁺¹ or ⁺² ions. It is believed that several copper dependent enzymes, mainly amine oxidases, are important components during wound healing. As another example, copper can be an integral component in angiogenesis or blood vessel formation. It is believed that copper stimulation of factors involved in vessel formation and maturation, such as vascular endothelial growth factor (VEGF), is mainly responsible for its angiogenesis effect.

Accordingly, in other aspects, the present invention provides methods for treating a wound 24. In one example, such a method comprises applying the textile material of the present disclosure to the wound 24. In some implementations of the first embodiment, applying the textile material of the present disclosure to the wound includes applying the textile material at or in the vicinity of the wound.

In accordance with an example of a suitable method, multiples of the wound dressings 10 may be manufactured substantially simultaneously, wherein the pads 12 may initially be part of a first precursor textile (not shown) and the backings 14 may initially be part of a second precursor textile (not shown). The first and second precursor textiles may then be laminated to one another by way of the above-discussed thermal lamination process to form a laminate from which the wound dressings 10 are cut. For example, the laminating may comprise the first and second precursor textiles being together nipped between at least a relatively hot calendar roll and a relatively cool calendar roll, with the relatively cool calendar roll engaging the outer surface of the first precursor web in order to substantially maintain the loft of the pads 12. In one example, in top and bottom plan views, the wound dressings 10, and thus the pads 12 and backings 14, may be four inch by four inch squares. A variety of differently sized and shaped wound dressings 10 are within the scope of this invention. The wound dressings 10 may be packaged and sterilized in a conventional manner.

In some implementations of the first embodiment, the wound dressing may also include other substances such as, for example, therapeutically beneficial substances including, but not limited to, antibiotics, vitamins, trophic factors, extracellular matrices, enzymes, enzyme inhibitors, defensins, polypeptides, anti-infective agents, buffering agents, minerals, analgesics, anticoagulants, coagulation factors, anti-inflammatory agents, vasoconstrictors, vasodilators, and/or diuretics, and the like.

A specific example of a wound dressing 10 that has been prepared and evaluated is presented as follows:

A first precursor textile was formed from composite fibers 16 (or the like), obtained as Cu+STAT®. The composite fibers (or the like) each had a length of about 3 inches, a denier of about 2.2, included polyester as the base polymer material, and included copper in a range of from about 0.1% by weight to about 0.4% by weight, or in a range of from about 30 ppm to about 120 ppm. The first precursor textile was formed by needlepunching the composite fibers (or the like). The first and second precursor textiles were laminated to one another by way of the above-discussed thermal lamination process to form a laminate from which the wound dressings were cut.

The resulting wound dressings 10, when tested, did not elicit a sensitization response under the conditions of an International Organization for Standardization (ISO) guinea pig maximization sensitization test. They met the requirements of the ISO acute systemic injection test, the requirements of the ISO intra-cutaneous reactivity test, and were considered non-pyrogenic in the materials mediated rabbit pyrogen test. These wound dressings 10 were further tested under the American Association of Textile Chemists and Colorists (AATCC) test method 100, as specified in the Good Laboratory Practices regulations. These wound dressings 10 were shown to be effective in reducing the bacterial populations by greater than 99.99% of Acinetobacter baumannii, Enterococcus faecalis (VRE), Escherichia coli, Pseudomonas aeruginosa, Methicillin-Resistant Staphylococcus aureus (MRSA), and Streptococcus pyogenes. These wound dressings 10 also showed a greater than 99.99% reduction in Trichophyton mentagrophytes, and a 98% reduction in Candida albicans, both fungal organisms.

Other embodiments are within the scope of the invention. For example, the pad 12 may be a woven fabric comprising the composite fibers 16 (e.g., in the form of yarns) and/or the backing 14 may be a film or a woven fabric, either of which may be apertured. As other examples, pads 12 in any suitable sizes and shapes may be used as wound dressings without being previously mounted to any backings 14, or the like. As further examples, the wound dressings 10 may include additional materials such as, but not limited to, foam, adhesive material and/or other suitable materials that those of ordinary skill in the art would know to include in wound dressings.

As another example, a second embodiment of this invention is like the first embodiment, except for variations noted and variations that will be apparent to one of ordinary skill in the art. Due to the similarity, components of the second embodiment that are identical, similar and/or function in at least some ways similarly to corresponding components of the first embodiment have reference numbers incremented by 100.

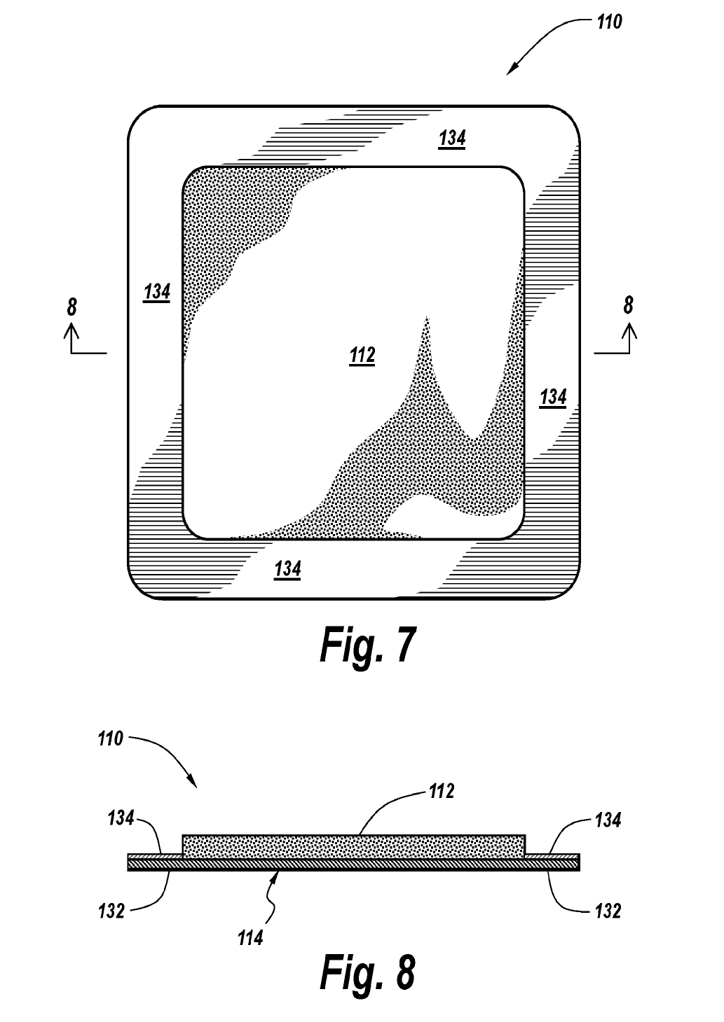

FIGS. 7 and 8 illustrate a wound dressing 110 of the second embodiment. In the second embodiment, the substrate or backing 114 is larger than the pad 112, so that at least one marginal portion 132 of the backing extends outwardly beyond a peripheral portion of the pad. A conventional pressure-sensitive adhesive material 134 may be carried by the marginal portion 132 of the backing 114. The adhesive material 134 may be a type of adhesive that is conventionally used in medical adhesive tapes, bandages, and the like, for mounting the wound dressing 110 to animal tissue. Each of the marginal portions 132 of the backing 114 and the adhesive 134 may extend at least partially around, or completely around, the periphery of the pad 112. A release liner (not shown) may be applied to the outer surface of the adhesive material 134 during manufacturing, and may be removed from the wound dressing 110 immediately prior to use of the wound dressing.

It will be understood by those skilled in the art that while the present invention has been discussed above with reference to exemplary embodiments, various additions, modifications and changes can be made thereto without departing from the spirit and scope of the invention, some aspects of which are set forth in the following claims.

What is claimed is:

1. A wound dressing, comprising:
a substrate; and
a textile material mounted to the substrate, the textile material comprising fibers and copper, wherein at least some of the copper is proximate exterior surfaces of at least some of the fibers; and
wherein the copper is present in an effective amount to stimulate wound healing and to restrict growth of microorganisms.

2. The wound dressing of claim 1, wherein the textile material is a pad that is thicker than the substrate.

3. The wound dressing of claim 1, wherein the textile material is a nonwoven textile fabric.

4. The wound dressing of claim 3, wherein the nonwoven textile fabric is a needled nonwoven textile fabric.

5. The wound dressing of claim 3, wherein the substrate is an apertured substrate.

6. The wound dressing of claim 3, wherein the substrate is an apertured nonwoven textile fabric.

7. The wound dressing of claim 1, wherein:
the substrate is a backing;
at least one marginal portion of the backing extends outwardly beyond a periphery of the textile material;
the wound dressing further comprises adhesive material on the at least one marginal portion of the backing; and
the adhesive material is configured for at least partially mounting the wound dressing to animal tissue.

8. The wound dressing of claim 7, wherein:
the at least one marginal portion of the backing extends at least partially around the textile material; and
the adhesive material extends at least partially around the textile material.

9. The wound dressing of any preceding claim, wherein the textile material is laminated to the substrate.

10. A method for treating a wound, the method comprising applying the wound dressing of any preceding claim to the wound.

11. A wound dressing, comprising:
a nonwoven textile fabric comprising fibers and copper, wherein at least some of the copper is proximate exterior surfaces of at least some of the fibers, and the copper is present in an effective amount to stimulate wound healing and to restrict growth of microorganisms.

12. The wound dressing of claim 11, wherein the nonwoven textile fabric is a needled nonwoven textile fabric.

13. The wound dressing of claim 11, further comprising a substrate mounted to the nonwoven textile fabric.

14. The wound dressing of claim 13, wherein the nonwoven textile fabric is thicker than the substrate.

15. The wound dressing of claim 13, wherein the substrate is an apertured substrate.

16. The wound dressing of claim 13, wherein the substrate is an apertured nonwoven textile fabric.

17. The wound dressing of claim 13, wherein:
the substrate is a backing;
at least one marginal portion of the backing extends outwardly beyond a periphery of the nonwoven textile fabric;
the wound dressing further comprises adhesive material on the at least one marginal portion of the backing; and
the adhesive material is configured for at least partially mounting the wound dressing to animal tissue.

18. The wound dressing of claim 17, wherein:
the at least one marginal portion of the backing extends at least partially around the nonwoven textile fabric; and
the adhesive material extends at least partially around the nonwoven textile fabric.

19. The wound dressing of any of claims 13 through 18, wherein the substrate is laminated to the nonwoven textile fabric.

20. A method for treating a wound, the method comprising applying the wound dressing of any of claims 11 through 19 to the wound.
